(19) United States
(12) Patent Application Publication
(10) Pub. No.: US 2008/0020076 A1
(43) Pub. Date: Jan. 24, 2008

(54) Methods and Therapies for Potentiating a Therapeutic Action of an Alpha-2 Adrenergic Receptor Agonist and Inhibiting and/or Reversing Tolerance to Alpha-2 Adrenergic Receptor Agonists

(76) Inventors: Khem Jhamandas, Kingston (CA); Brian Milne, Kingston (CA)

Correspondence Address: LICATA & TYRRELL P.C., 66 E. MAIN STREET, MARLTON, NJ, 08053, US

(21) Appl. No.: 11/780,504

(22) Filed: Jul. 20, 2007

Related U.S. Application Data
(60) Provisional application No. 60/832,470, filed on Jul. 21, 2006.

(51) Int. Cl. A61K 31/04; A61K 31/415; A61K 36/00

(52) U.S. Cl. 424/775; 514/398; 514/741

(57) ABSTRACT

Combination therapies of an alpha-2 adrenergic receptor agonist and an alpha-2 adrenergic receptor antagonist at a concentration effective to potentiate but not antagonize a therapeutic effect of the alpha-2 adrenergic receptor agonist are provided. Also provided are methods for use of these combination therapies in potentiating the therapeutic effects of alpha-2 adrenergic receptor agonists, inhibiting development of acute and/or chronic tolerance to alpha-2 adrenergic receptor agonists and treating conditions treatable by alpha-2 adrenergic receptor agonist therapy in a subject. In addition, a method for reversing alpha-2 adrenergic receptor agonist tolerance and/or restoring therapeutic effect of an alpha-2 adrenergic receptor agonist in a subject via administration of an alpha-2 adrenergic receptor antagonist at a concentration effective to potentiate, but not antagonize, the therapeutic effect of the alpha-2 adrenergic receptor agonist is provided.

This patent application claims the benefit of priority from U.S. Provisional Application Ser. No. 60/832,470, filed Jul. 21, 2006, teachings of which are herein incorporated by reference in their entirety.

### BACKGROUND OF THE INVENTION

L-norepinephrine is a major transmitter in the pathways descending from the brainstem nuclei to the spinal dorsal horn, a region involved in the transfer and processing of noxious input. At the spinal cord level, norepinephrine acts as an agonist on the alpha-2 adrenergic receptors to depress activity of nociceptive neurons transmitting pain signals from periphery to the brain. Activation of the alpha-2 adrenergic receptors inhibits the release of pain transmitters such as substance P from nociceptive neurons. In addition, activation of alpha-2 adrenergic receptors inhibits (hyperpolarizes) projection neurons that receive the noxious input and convey this input to specific brain areas.

At the spinal level, alpha-2 adrenergic receptors and opioid receptors have similar anatomical representation and their respective agonists produce effects via common cellular mechanisms.

However, the use of spinal alpha-2 adrenergic receptor agonists such as clonidine for spinal analgesia produces adverse effects such as sedation and/or hypotension. Further, the repeated exposure to the spinally injected alpha-2 adrenergic receptor agonists produces tolerance and physical dependence. These factors have limited therapeutic application of the alpha-2 adrenergic receptor agonists in the treatment of pain.

Combination therapies for reducing the amount of alpha-2 adrenergic receptor agonist required to provide analgesia have been described.

WO 98/38997 discloses use of levobupivacaine and an opioid or alpha-2 adrenergic receptor agonist in a medicament for anesthesia and analgesia.

The actions of alpha-2 adrenergic receptor agonists are blocked by atipemazole and yohimbine. Atipemazole is a potent, selective and specific antagonist of both centrally and peripherally located alpha-2 adrenoceptors that is about 100 times more potent as a displacer of clonidine than yohimbine (Virtanen et al. Arch. Int. Pharmacodyn. 1989 297:190-204).

Browning et al. disclosed that the alpha-2 adrenergic receptor agonist analgesic activity was antagonized by alpha-2 adrenergic receptor antagonists (Br. J. Pharmacol. 1982 77:487-491).

Accordingly, there is a need for therapies potentiating the therapeutic effects of alpha-2 adrenergic receptor agonist activities, particularly their analgesic activity while limiting their unwanted side effects.

### SUMMARY OF THE INVENTION

An aspect of the present invention is a composition comprising an alpha-2 adrenergic receptor agonist, at a concentration effective to produce a therapeutic effect, and an alpha-2 adrenergic receptor antagonist, at a concentration effective to potentiate, but not antagonize the therapeutic effect of the alpha-2 adrenergic receptor agonist. Compositions of the present invention provide useful therapeutic agents for management of pain including, but not limited to, management of chronic and/or acute pain and/or neuropathic pain and/or nociceptive pain, e.g. acute post-surgical and/or peri-operative pain, obstetrical pain including labor as well as pain associated with caesarean section, post amputation pain, pain associated with conditions such as sympathetic dystrophy, neuralgia, arthritis, fibromyalgia and cancer, pain in children, lower back pain and as an adjunct to peripheral nerve blocks. For pain management the alpha-2 adrenergic receptor agonist is preferably administered via epidural. Compositions of the present invention are also useful in treating hypertension, glaucoma, nasal congestion, anxiety and opioid withdrawal symptoms. The alpha-2 adrenergic receptor agonist may be administered as a secondary or tertiary drug for treatment of any of the above conditions.

Another aspect of the present invention is a method for potentiating a therapeutic effect of an alpha-2 adrenergic receptor agonist which comprises administering to a subject in combination with an alpha-2 adrenergic receptor agonist an alpha-2 adrenergic receptor antagonist at a concentration effective to potentiate, but not antagonize, the therapeutic effect of the alpha-2 adrenergic receptor agonist. By potentiating the therapeutic effect of the alpha-2 adrenergic receptor agonist, a lower concentration of alpha-2 adrenergic receptor agonist may be administered thereby alleviating unwanted side effects associated with treatment of alpha-2 adrenergic receptor agonists.

Another aspect of the present invention is a method for potentiating a biological action of an endogenous alpha-2 adrenergic receptor agonist in a subject which comprises administering to the subject an alpha-2 adrenergic receptor antagonist at a concentration effective to potentiate, but not antagonize, the biological action of the endogenous alpha-2 adrenergic receptor agonist.

Another aspect of the present invention is a method for inhibiting development of acute tolerance to a therapeutic action of an alpha-2 adrenergic receptor agonist in a subject which comprises administering to a subject in combination with an alpha-2 adrenergic receptor agonist an alpha-2 adrenergic receptor antagonist at a concentration effective to potentiate, but not antagonize the therapeutic effect of the alpha-2 adrenergic receptor agonist.

Another aspect of the present invention is a method for inhibiting development of chronic tolerance to a therapeutic action of an alpha-2 adrenergic receptor agonist in a subject which comprises administering to a subject in combination with an alpha-2 adrenergic receptor agonist an alpha-2 adrenergic receptor antagonist at a concentration effective to potentiate, but not antagonize the therapeutic effect of the alpha-2 adrenergic receptor agonist.

Another aspect of the present invention is a method for reversing tolerance to a therapeutic action of an alpha-2 adrenergic receptor agonist and/or restoring therapeutic potency of an alpha-2 adrenergic receptor agonist in a subject tolerant to a therapeutic action of an alpha-2 adrenergic receptor agonist which comprises administering an alpha-2 adrenergic receptor antagonist to a subject receiving an alpha-2 adrenergic receptor agonist at a concentration effective to potentiate, but not antagonize, the therapeutic effect of the alpha-2 adrenergic receptor agonist.

Another aspect of the present invention is a method for treating a subject suffering from a condition treatable with an alpha-2 adrenergic receptor agonist comprising administering to the subject an alpha-2 adrenergic receptor agonist at a concentration effective to produce a therapeutic effect and an alpha-2 adrenergic receptor antagonist at a concentration effective to potentiate, but not antagonize, the therapeutic effect of the alpha-2 adrenergic receptor agonist. This method is useful in treating subjects suffering from conditions including, but not limited to, pain, hypertension, glaucoma, nasal congestion, anxiety and opioid withdrawal symptoms.

Yet a further aspect of the present invention in each of the above methods is that the action or treatment occurs without substantial side effects.

### BRIEF DESCRIPTION OF THE FIGURES

FIGS. 1A and 1B are line graphs showing the effects of the alpha-2 adrenergic receptor antagonist atipemazole at inhibiting analgesia by the alpha-2 adrenergic receptor agonist clonidine in the tail-flick test (FIG. 1A) and paw pressure test (FIG. 1B) in rats. Clonidine was administered intrathecally at 200 nmoles (53 micrograms). Rats were co-administered atipemazole intrathecally at 0 micrograms (open circle), 1 microgram (filled square), 5 micrograms (filled triangle), and 10 micrograms (inverted filled triangle).

FIGS. 2A-2F are line graphs showing the effects of ultra-low doses of alpha-2 adrenergic receptor antagonists potentiating analgesia by alpha-2 adrenergic receptor agonists. FIGS. 2A and 2B show the effects of an ultra-low dose of alpha-2 adrenergic receptor antagonist atipemazole at potentiating analgesia of L-norepinephrine in the tail-flick test (FIG. 2A) and paw pressure test (FIG. 2B) in rats. L-norepinephrine was administered intrathecally at 30 μg. Rats were co-administered atipemazole intrathecally at 0 micrograms (open circle) and 0.08 ng (inverted filled triangle). FIGS. 2C and 2D are line graphs showing the effects of an ultra-low dose of alpha-2 adrenergic receptor antagonist atipemazole at potentiating analgesia by the alpha-2 adrenergic receptor agonist clonidine in the tail-flick test (FIG. 2C) and paw pressure test (FIG. 2D) in rats. Clonidine was administered intrathecally at 13.3 μg. Rats were co-administered atipemazole intrathecally at 0 micrograms (open square), 0.0008 ng (filled square), 0.008 ng (filled circle), 0.08 ng (filled inverted triangle), 0.8 ng (filled triangle) and 8 ng (filled diamond). FIGS. 2E and 2F are line graphs showing the effects of an ultra-low dose of alpha-2 adrenergic receptor antagonist yohimbine at potentiating analgesia by the alpha-2 adrenergic receptor agonist clonidine in the tail-flick test (FIG. 2E) and paw pressure test (FIG. 2F) in rats. Clonidine was administered intrathecally at 13.3 μg. Rats were co-administered yohimbine intrathecally at 0 micrograms (open square), 0.02 ng (filled triangle), 0.005 ng (filled inverted triangle), and 0.0002 ng (filled diamond). Rats administered saline alone (20 μl) are depicted by “X”.

FIGS. 3A and 3B are line graphs showing the effects of the alpha-2 adrenergic receptor antagonist atipemazole administered at a dose ineffective at causing alpha-2 adrenergic receptor blockade on acute tolerance to the analgesic actions of spinal. L-norepinephrine in the tail flick test (FIG. 3A) and paw pressure test (FIG. 3B) in rats. In this study, acute tolerance was produced by delivering three intrathecal successive injections of (30 μg) at 90 minute intervals (depicted by open circles). Additional groups of rats received a combination of L-norepinephrine and a fixed dose of atipemazole at 0.8 ng (depicted by filled diamonds) or 0.008 ng (depicted as filled inverted triangles). The effects of normal saline (20 μl) (depicted as X) were also evaluated by injection at 90 minute intervals.

FIGS. 4A and 4B are cumulative dose-response curves (DRCs) for the acute analgesic action of intrathecal L-norepinephrine, in the four treatment groups of FIGS. 3A and 3B, derived 24 hours after the first L-norepinephrine injection. Rats administered L-norepinephrine alone are depicted by open circles. Rats administered L-norepinephrine and atipemazole at 0.8 ng are depicted by filled diamonds. Rats administered L-norepinephrine and atipemazole at 0.008 ng are depicted by filled inverted triangles. Rats administered saline are depicted by X.

FIGS. 5A and 5B are bar graphs showing the ED₅₀ values (effective dose in 50% of the animals), an index of potency, derived from the cumulative dose-response curves of FIGS. 4A and 4B, respectively. Rats administered L-norepinephrine alone are depicted by the dotted bar. Rats administered L-norepinephrine and atipemazole at 0.8 ng and 0.008 ng are depicted by the horizontal and vertical lined bars, respectively. Rats administered saline are depicted by the unfilled bar.

### DETAILED DESCRIPTION OF THE INVENTION

It has now been found that administration of ultra-low doses of an alpha-2 adrenergic receptor antagonist potentiates alpha-2 adrenergic receptor agonist analgesia and inhibits the development of acute tolerance to alpha-2 adrenergic receptor agonists. The present invention provides new combination therapies for potentiating therapeutic activities of an alpha-2 adrenergic receptor agonist and inhibiting development of and/or reversing acute tolerance to an alpha-2 adrenergic receptor agonist involving co-administration of an alpha-2 adrenergic receptor agonist with an alpha-2 adrenergic receptor antagonist. An aspect of the present invention is compositions comprising an alpha-2 adrenergic receptor agonist and an ultra-low dose of an alpha-2 adrenergic receptor antagonist. Another aspect of the present invention is methods for potentiating a therapeutic action of an alpha-2 adrenergic receptor agonist and/or effectively inhibiting the development of acute as well as chronic tolerance to a therapeutic action of an alpha-2 adrenergic receptor agonist by co-administering the alpha-2 adrenergic receptor agonist with an ultra-low dose of an alpha-2 adrenergic receptor antagonist. The new combination therapies of the present invention are expected to be useful in optimizing the use of alpha-2 adrenergic receptor agonist drugs in various applications including but not limited to: management of chronic and/or acute pain and/or neuropathic pain and/or nociceptive pain, e.g. management of acute post-surgical and/or peri-operative pain, obstetrical pain including labor as well as pain associated with caesarean section, post amputation pain, pain associated with conditions such as sympathetic dystrophy, neuralgia, arthritis, fibromyalgia and cancer, pain in children, and lower back pain, as an adjunct to peripheral nerve blocks, hypertension, glaucoma, nasal congestion, anxiety and opioid withdrawal symptoms. In a preferred embodiment, the combination therapies of the present invention are used in pain management. For pain management the alpha-2 adrenergic receptor agonist is preferably administered epidurally.

Alpha-2 adrenergic receptor antagonists useful in the combination therapies and methods of the present invention include any compound that partially or completely reduces, inhibits, blocks, inactivates and/or antagonizes the binding of an alpha-2 adrenergic receptor agonist to its receptor to any degree and/or the activation of an alpha-2 adrenergic receptor to any degree. Thus, the term alpha-2 adrenergic receptor antagonist is also meant to include compounds that antagonize the agonist in a competitive, irreversible, pseudo-irreversible and/or allosteric mechanism. In addition, the term alpha-2 adrenergic receptor antagonist includes compounds at low dose or ultra-low doses that increase, potentiate and/or enhance the therapeutic and/or analgesic potency and/or efficacy of the alpha-2 adrenergic receptor agonists, while at similar doses may not demonstrate a substantial or significant antagonism of an alpha-2 adrenergic receptor agonists. Examples of alpha-2 adrenergic receptor antagonists useful in the combination therapies and methods of the present invention include, but are in no way limited to atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine. Additional examples of agents which exhibit some alpha-2 and/or alpha-1 receptor antagonistic activity and thus may be useful in the present invention include, but are not limited to, venlafaxine, doxazosin, phentolamine, dihydroergotamine, ergotamine, phenothiazines, phenoxybenzamine, piperoxane, prazosin, tamsulosin, terazosin, and tolazoline.

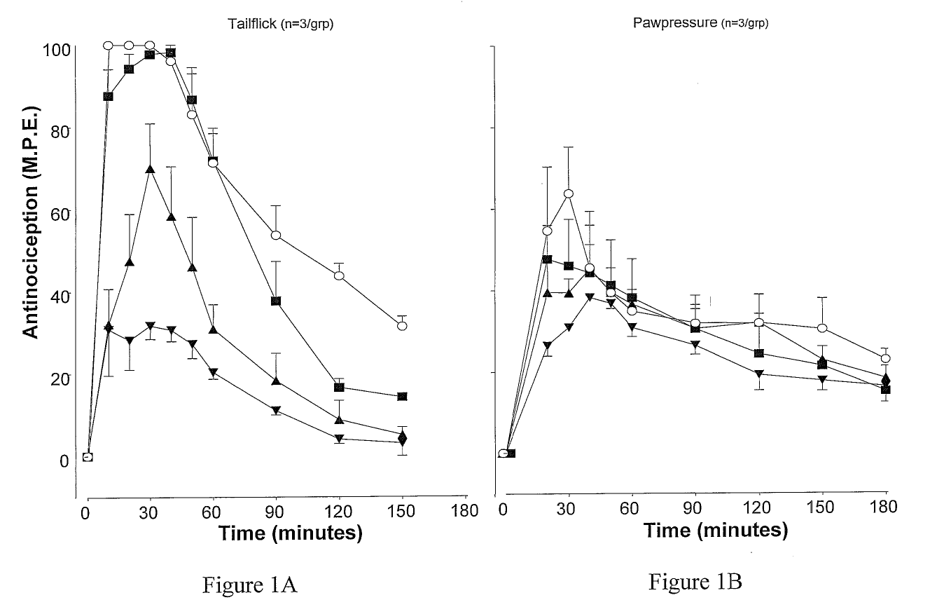

The alpha-2 adrenergic receptor antagonist is included in the compositions and administered in the methods of the present invention at an ultra-low dose. By “ultra-low” dose as used herein it is meant a concentration of alpha-2 adrenergic receptor antagonist that potentiates, but does not antagonize, a therapeutic effect of the alpha-2 adrenergic receptor agonist. Thus, in one embodiment, by the term “ultra-low dose” it is meant an amount or concentration of the alpha-2 adrenergic receptor antagonist lower than that established by those skilled in the art to significantly block or inhibit alpha-2 adrenergic receptor agonist activity. For example, in one embodiment, by ultra-low dose of alpha-2 adrenergic receptor antagonist it is meant a concentration ineffective at alpha-2 adrenergic receptor blockade as measured in experiments such as set forth in FIGS. 1A and 1B. As will be understood by the skilled artisan upon reading this disclosure, however, other means for measuring alpha-2 adrenergic receptor antagonism can be used. Based upon these experiments, ultra-low doses of atipemazole which potentiate the therapeutic action of analgesia of the alpha-2 adrenergic receptor agonist norepinephrine were identified as being 12,000-fold to 1,200,000-fold lower than the dose producing a blockade of the spinal alpha-2 adrenergic receptors, as evidenced by antagonism of intrathecal clonidine (alpha-2 agonist) analgesia (FIG. 1A and FIG. 1B). Ultra-low doses useful in the present invention for other alpha-2 adrenergic receptor antagonists as well as other therapeutic actions of alpha-2 adrenergic receptor agonists can be determined routinely by those skilled in the art in accordance with the known effective concentrations as alpha-2 adrenergic receptor blockers and the methodologies described herein for atipemazole. In general, however, by “ultra-low” it is meant a dose at least 1,000- to 1,000,000-fold lower that the maximal dose producing a blockade of alpha-2 adrenergic receptors.

By “ultra-low dose” it is also meant to be inclusive of a concentration of alpha-2 adrenergic receptor antagonist which is significantly less than the concentration of alpha-2 adrenergic receptor agonist to be administered. For example, the ultra-low dose of alpha-2 adrenergic receptor antagonist can be expressed as a ratio with respect to the dose of alpha-2 adrenergic receptor agonist administered or to be administered. A preferred ratio for an ultra-low dose is a ratio of 1:1,000, 1:10,000, 1:100,000 or 1:1,000,000 or any ratio in between of alpha-2 adrenergic receptor antagonist to alpha-2 adrenergic receptor agonist.

Another preferred “ultra-low” dose is a concentration or ratio which potentiates the therapeutic action of the alpha-2 adrenergic receptor agonist while alleviating, inhibiting, preventing or diminishing an unwanted side effect or side effects. For example, for pain management with an alpha-2 adrenergic receptor agonist such as clonidine, administration of an ultra low dose of an alpha-2 adrenergic receptor antagonist in accordance with the present invention will lessen adverse effects of clonidine administration such as sedation and/or hypotension.

Alpha-2 adrenergic receptor agonists useful in the combination therapies and methods of the present invention include any compound that binds to and/or activates and/or agonizes at least one or more alpha-2 adrenergic receptor subtypes to any degree and/or stabilizes at least one or more alpha-2 adrenergic receptor subtypes in an active or inactive conformation. Thus, by the term alpha-2 adrenergic receptor agonist it is meant to include partial agonists, inverse agonists, as well as complete agonists of one or more alpha-2 adrenergic receptor subtypes. Preferred alpha-2 adrenergic receptor agonists include those compounds which act as analgesics. Examples of alpha-2 adrenergic receptor agonists useful in the present invention include, but are in no way limited to L-norepinephrine, clonidine, dexmetdetomidine, apraclonidine, tizanidine, brimonidine, xylometazoline, tetrahydrozoline, oxymetazoline, guanfacine, guanabenz, xylazine, moxonidine, rilmenidine, UK 14,304, B-HT 933, B-HT 920, and octopamine. The concentration of alpha-2 adrenergic receptor agonist included in the compositions of the present invention and used in the methodologies described herein is a concentration effective to produce a therapeutic effect. Thus, by effective concentration as used herein it is meant an amount of alpha-2 adrenergic receptor agonist well known to the skilled artisan as having a therapeutic action or effect in a subject. Dosages of alpha-2 adrenergic receptor agonist, e.g. clonidine, producing, for example, an analgesic effect in human patients upon epidural bolus administration can typically range between about 150 μg to 800 μg and 3-12 μg/hour by epidural infusion. However, as will be understood by the skilled artisan upon reading this disclosure, the effective concentration will vary depending upon the alpha-2 adrenergic receptor agonist selected, the route of administration, the frequency of administration, the formulation administered, and the condition being treated. Further, as demonstrated herein, co-administration of an alpha-2 adrenergic receptor agonist with an ultra-low dose of an alpha-2 adrenergic receptor antagonist potentiates the analgesic effect of the alpha-2 adrenergic receptor agonist. Thus, by effective concentration as used herein it is also meant to include a lower amount or dose of alpha-2 adrenergic receptor agonist effective at producing a therapeutic effect when co-administered with an alpha-2 adrenergic receptor antagonist in accordance with the present invention, than when administered alone. It is expected that administration of these lower therapeutically effective amounts will be advantageous in alleviating unwanted side effects, including, but not limited to, development of physical dependence, of alpha-2 adrenergic receptor agonists.

For purposes of the present invention, by “therapeutic effect” or “therapeutic activity” or “therapeutic action” it is meant a desired pharmacological activity of an alpha-2 adrenergic receptor agonist useful for the treatment of a condition routinely treated with an alpha-2 adrenergic receptor agonist. Examples of such conditions include, but are not limited to, pain, hypertension, glaucoma, nasal congestion, anxiety and opioid withdrawal symptoms. By “treatment” of these conditions it is meant to include the inhibition, reduction or prevention of the condition as well as the inhibition, reduction or prevention of symptoms associated with the condition, and may result from the alpha-2 adrenergic receptor agonist inhibiting or preventing an undesired action associated with the condition or the alpha-2 adrenergic receptor agonist enhancing a desired action associated with the condition. By these terms it is meant to include a pharmacological activity or therapeutic outcome measurable as an end result, i.e. alleviation of pain or nasal congestion, as well as a pharmacological activity associated with a mechanism of action linked to the end desired result. In a preferred embodiment, the “therapeutic effect” or “therapeutic activity” or “therapeutic action” is alleviation or management of pain.

For purposes of the present invention, by “potentiate”, it is meant that administration of the alpha-2 adrenergic receptor antagonist enhances, extends or increases the therapeutic activity of an alpha-2 adrenergic receptor agonist and/or results in a decreased amount of alpha-2 adrenergic receptor agonist being required to produce a therapeutic effect. Thus, as will be understood by the skilled artisan upon reading this disclosure, the effective concentrations of alpha-2 adrenergic receptor agonist included in the combination therapies of the present invention may be decreased as compared to an established effective concentration for the alpha-2 adrenergic receptor agonist when administered alone. The amount of the decrease for other alpha-2 adrenergic receptor agonists can be determined routinely by the skilled artisan based upon ratios described herein for L-norepinephrine and atipemazole. By potentiate it is also meant to include any enhancement, extension or increase in therapeutic activity of an endogenous alpha-2 adrenergic receptor agonist in a subject upon administration of an ultra-low dose of an alpha-2 adrenergic receptor antagonist.

This decrease in required alpha-2 adrenergic receptor agonist to achieve the same therapeutic benefit will decrease any unwanted side effects associated with alpha-2 adrenergic receptor agonist therapy. Thus, the combination therapies of the present invention also provide a means for decreasing unwanted side effects of alpha-2 adrenergic receptor agonist therapy.

Enhancing endogenous alpha-2 adrenergic receptor agonist activity, and in particular norepinephrine may be useful in potentiating treatment of other drugs which act, at least in part, through endogenous release of norepinephrine. Examples of such drugs include, but are not limited to antidepressants such as monoamine oxidase inhibitors, venlafaxine, reboxitine and tricyclics such as amitriptyline, analgesics such as tramadol, and stimulants such as amphetamine and methylphenidate.

By “antagonize” as used herein, it is meant an inhibition or decrease in therapeutic effect or action of an alpha-2 adrenergic receptor agonist resulting from addition of an alpha-2 adrenergic receptor antagonist which renders the alpha-2 adrenergic receptor agonist ineffective or less effective therapeutically for the condition being treated.

By “tolerance” as used herein, it is meant a loss of drug potency and is produced by many alpha-2 adrenergic receptor agonists, and particularly norepinephrine. Chronic or acute tolerance can be a limiting factor in the clinical use of alpha-2 adrenergic receptor agonists as potency is decreased upon exposure to the alpha-2 adrenergic receptor agonist. By “chronic tolerance” it is meant a decrease in potency which can develop after drug exposure over several or more days. “Acute tolerance” is a loss in drug potency which can develop after drug exposure over several hours (Fairbanks and Wilcox J. Pharmacol. Exp. Therapeutics. 1997 282:1408-1417; Kissin et al. Anesthesiology 1991 74:166-171). Loss of alpha-2 adrenergic receptor agonist potency may also be seen in pain conditions such as neuropathic pain without prior exposure as neurobiological mechanisms underlying the genesis of tolerance and neuropathic pain are similar (Mao et al. Pain 1995 61:353-364). This is also referred to as acute tolerance. Tolerance has been explained in terms of alpha-2 adrenergic receptor desensitization. It has also been explained on the basis of an adaptive increase in levels of pain transmitters such as L-glutamic acid, substance P or CGRP. Inhibition of tolerance and maintenance of alpha-2 adrenergic receptor agonist potency are important therapeutic goals in pain management which, as demonstrated herein, are achieved via the combination therapies of the present invention.

One skilled in the art would know which combination therapies would work to potentiate a therapeutic action of an alpha-2 adrenergic receptor agonist and/or inhibit acute or chronic alpha-2 adrenergic receptor agonist tolerance upon co-administration of an ultra-low dose of an alpha-2 adrenergic receptor antagonist based upon the disclosure provided herein. For example, any given combination of alpha-2 adrenergic receptor agonist and alpha-2 adrenergic receptor antagonist may be tested in animals using one or more available tests, including, but not limited to, tests for analgesia such as thermal, mechanical and the like, or any other tests useful for assessing antinociception as well as other therapeutic actions of alpha-2 adrenergic receptor agonists. Non-limiting examples for testing acute analgesia include the thermal rat tail flick and mechanical rat paw pressure antinociception assays.

The ability of exemplary combination therapies of the present invention to potentiate the analgesic action of an alpha-2 adrenergic receptor agonist and/or inhibit acute alpha-2 adrenergic receptor agonist tolerance upon co-administration of an ultra-low dose of an alpha-2 adrenergic receptor antagonist was demonstrated in tests of both thermal (rat tail flick) and mechanical (rat paw pressure) antinociception. In these experiments, alpha-2 adrenergic receptor antagonists used were atipemazole and yohimbine. The alpha-2 adrenergic receptor agonists were L-norepinephrine and clonidine.

Atipemazole administered intrathecally antagonizes the analgesic action of the alpha-2 adrenergic receptor agonist clonidine at doses greater than 1 microgram. FIGS. 1A and 1B show the effects of atipemazole on the clonidine-induced analgesia in the tail flick (FIG. 1A) and paw pressure test (FIG. 1B). Injection of clonidine (200 nmoles), an alpha-2 adrenergic receptor agonist, produced a maximal analgesic response in the tail flick test and a lesser effect in the paw pressure test. Co-administration of three different doses of atipemazole produced a dose-related decrease in the peak clonidine analgesia in the tail flick test, the highest drug dose (10 μg) almost abolishing the response. Atipemazole also decreased clonidine response in the paw pressure test but only at the highest dose. These experiments established that the atipemazole could block clonidine analgesia, an effect consistent with its identity as an alpha-2 adrenergic receptor antagonist.

Thus, for all subsequent tests involving atipemazole interactions with norepinephrine, the atipemazole dose was lowered to the exemplary ultra-low doses of 0.8 ng, 0.08 ng and 0.008 ng, representing a 12,000-fold to 1,200,000-fold decrease in the dose producing maximal alpha-2 adrenergic receptor blockade.

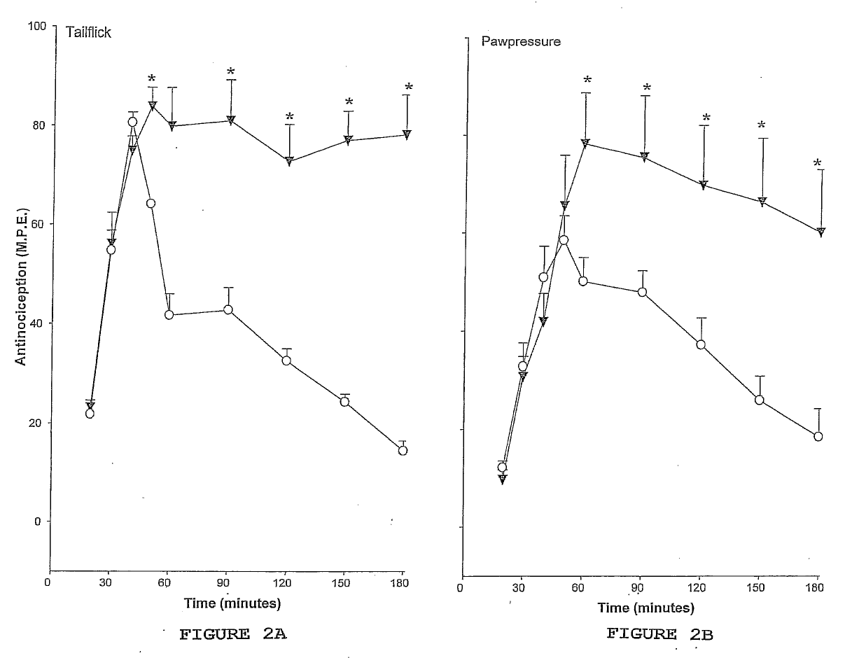

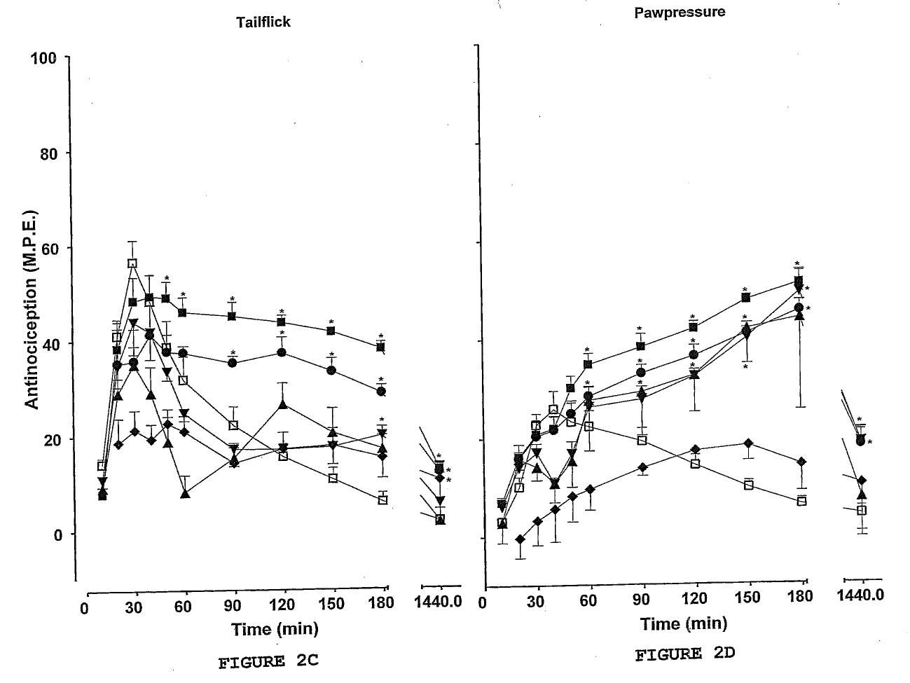

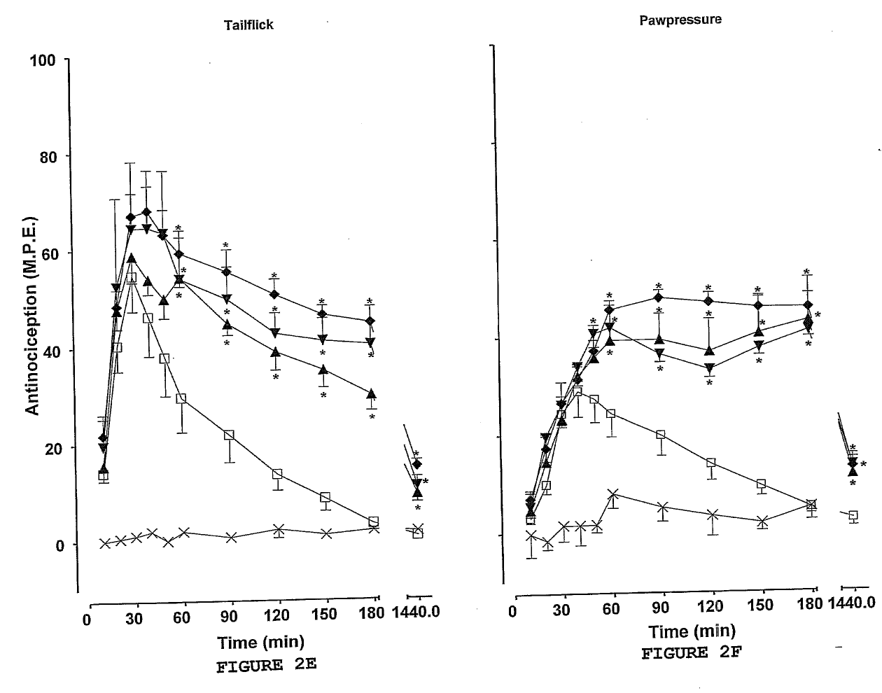

As shown in FIGS. 2A and 2B, administration of a single spinal dose of the alpha-2 adrenergic receptor agonist norepinephrine produced analgesia that peaked at 30 minutes and terminated at 180 minutes. Addition of the ultra-low dose of the alpha-2 adrenergic receptor antagonist, atipemazole (0.08 ng), extended norepinephrine analgesia both in the tail flick test (FIG. 2A) and the paw pressure test (FIG. 2B). The dose of atipemazole used in these experiments is several thousand fold lower than the dose that has been shown to block the spinal analgesia produce by the alpha-2 adrenergic receptor agonist, clonidine.

The effects of ultra-low doses of atipemazole were also examined on the action of clonidine which acts as a selective alpha-2 adrenergic receptor agonist. As shown in FIG. 2C and FIG. 2D administration of 50 nmoles of intrathecal clonidine produced a submaximal analgesic response in the tail flick and paw pressure test. This response was significantly augmented by combination of clonidine with 0.0008, 0.008, and 0.08 ng of atipemazole.

To establish that the effects of atipemazole could be replicated with another alpha-2 adrenergic receptor antagonist, the effects of yohimbine were tested on the clonidine-induced analgesia in the tailflick and paw pressure test. Clonidine (50 nmoles; 13.3 μg) produced a submaximal analgesia in the tail flick (FIG. 2E) and paw pressure test (FIG. 2F). This response was significantly augmented by combination of clonidine with the alpha-2 adrenergic receptor antagonist yohimbine (0.0002; 0.005; and 0.02 ng).

Thus, these experiments demonstrate that the analgesic effects of alpha-2 adrenergic receptor agonists can be potentiated by ultra-low doses of alpha-2 adrenergic receptor antagonists.

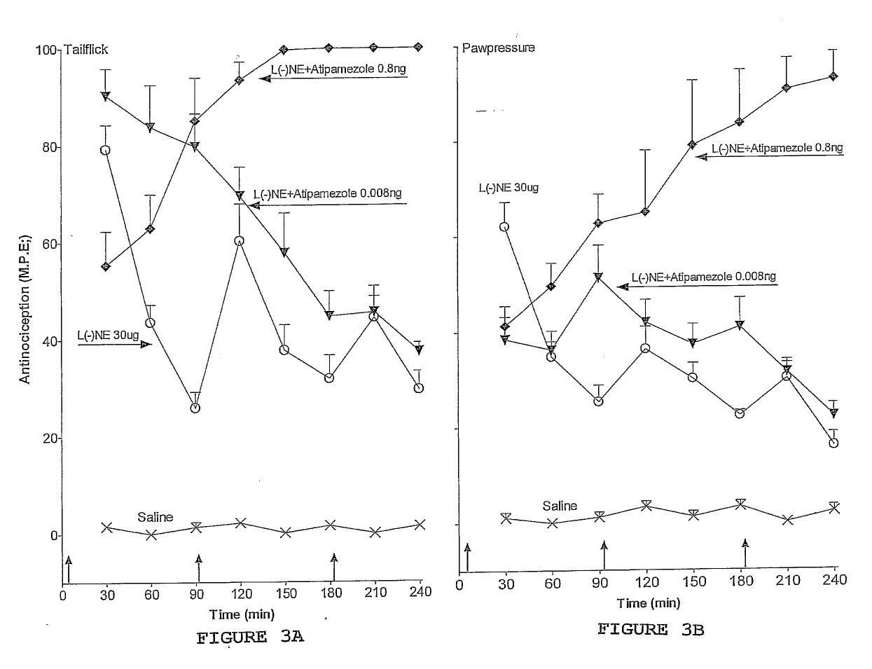

The effects of ultra-low doses of atipemazole on the development of acute tolerance to norepinephrine were also examined. The development of acute tolerance is indicated by a rapid decline of the analgesic effect following repeated administration of norepinephrine over several hours. In these experiments, acute tolerance was produced by delivering three intrathecal successive injections of L-norepinephrine (30 μg) at 90 minute intervals. In subsequent experiments, L-norepinephrine was combined with a fixed dose of atipemazole (0.8 ng or 0.008 ng). The effect of normal saline (20 μl) was also evaluated by injection at 90 minute intervals. Pain responses were evaluated in the tail flick and paw pressure test at 30 minute intervals. As shown in FIGS. 3A and 3B, co-administration of atipemazole (0.8 ng) with norepinephrine inhibited the decline of norepinephrine analgesia. At 240 minutes, after the third dose of norepinephrine had been administered, the analgesic response following administration of norepinephrine alone had significantly declined towards pre-drug baseline value. In contrast, administration of a combination of norepinephrine and atipemazole (0.8 ng) sustained the maximal analgesic level. Thus, atipemazole demonstrated the ability to prevent the acute tolerance to norepinephrine. Twenty-four hours after the drug treatment, cumulative dose-response curves (DRCS) for the action of L-norepinephrine in each treatment group were obtained to establish the drug potency index. This index, represented by the L-norepinephrine ED₅₀ (effective dose in 50% of animals tested) was calculated from the cumulative dose-response curves. Tolerance was indicated by a rightward shift in the L-norepinephrine dose-response curve and an increase in the L-norepinephrine ED₅₀ value. This rightward shift of the dose response curve was prevented in animals given combination of norepinephrine with atipemazole (0.008 or 0.8 ng).

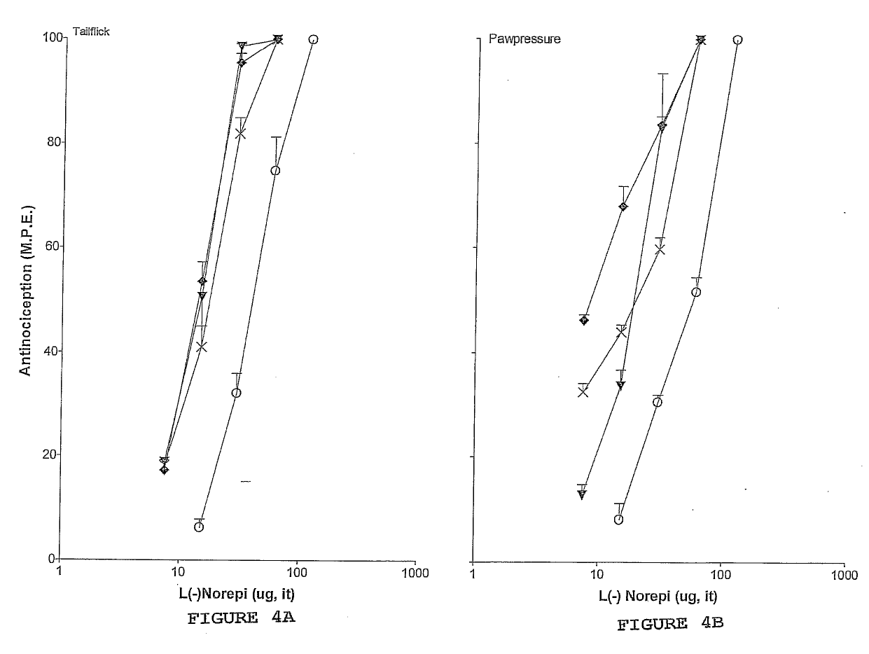

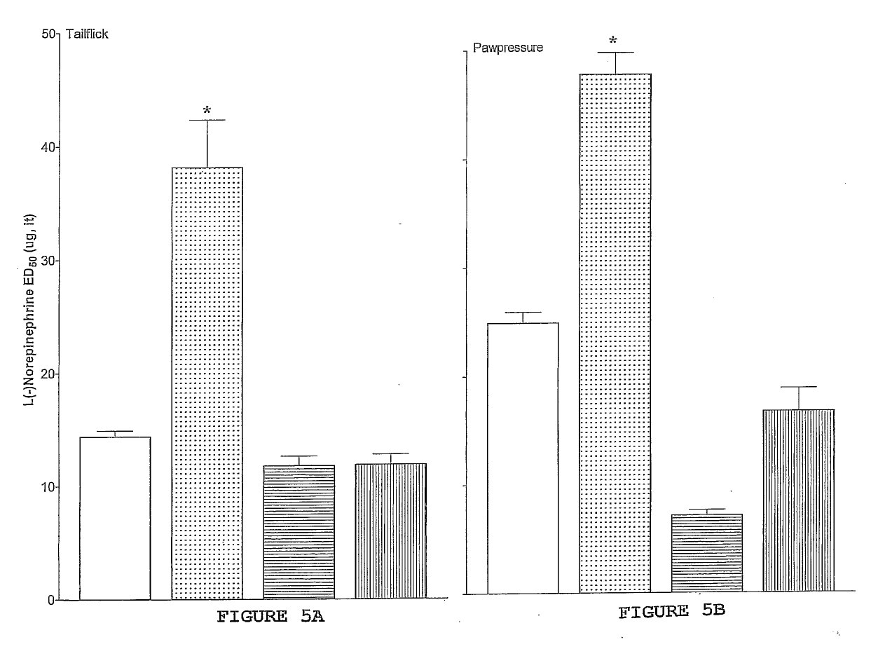

FIGS. 5A and 5B show the ED₅₀ values, reflecting potency of norepinephrine, which were derived from the dose response curves shown in FIGS. 4A and 4B, respectively. As shown, in the group that received three successive injections of norepinephrine (FIG. 3), the ED₅₀ value increased significantly over the values obtained in animals that had received repeated injections of saline (control group), reflecting a loss of norepinephrine potency as a result of repeated exposure. Ultra-low doses of atipemazole (0.008 or 0.8 ng) combined with norepinephrine completely prevented the increase in ED50 values. Thus, ultra-low dose atipemazole completely prevented the loss of norepinephrine potency associated with the development of acute tolerance to its analgesic action in the tailflick and paw pressure test.

Thus, as shown by these experiments ultra-low dose administration of an alpha-2 adrenergic receptor antagonist such as atipemazole very effectively potentiates the analgesic effect of an alpha-2 adrenergic receptor agonist such as L-norepinephrine and inhibits the development of acute tolerance to an alpha-2 adrenergic receptor agonist such as L-norepinephrine. Thus, these combination therapies of the present invention are useful in pain management in a subject.

Ultra-low dose atipemazole, when administered alone, is also expected to be useful in potentiating endogenous alpha-2 adrenergic receptor agonists such as norepinephrine. Thus, the present invention also provides methods for potentiating the therapeutic actions of endogenous alpha-2 adrenergic receptor agonists such as norepinephrine in a subject (not being administered an exogenous alpha-2 adrenergic receptor agonist) upon administration of an ultra-low dose alpha-2 adrenergic receptor antagonist to the subject.

As will be understood by the skilled artisan upon reading this disclosure, the present invention is not limited to the specific examples of potentiating alpha-2 adrenergic receptor agonist effects and inhibiting and/or reversing tolerance set forth herein, but rather, the invention should be construed and understood to include any combination of an alpha-2 adrenergic receptor agonist and alpha-2 adrenergic receptor antagonist wherein such combination has the ability to potentiate the effect of the alpha-2 adrenergic receptor agonist as compared to the effect of the alpha-2 adrenergic receptor agonist when used alone or to inhibit and/or reverse tolerance to an alpha-2 adrenergic receptor agonist therapy. Based on the teachings set forth in extensive detail elsewhere herein, the skilled artisan will understand how to identify such alpha-2 adrenergic receptor agonists, alpha-2 adrenergic receptor antagonists, and combinations thereof, as well as the concentrations of alpha-2 adrenergic receptor agonists and alpha-2 adrenergic receptor antagonists to use in such a combination useful in the present invention.

For pain management, alpha-2 adrenergic receptor agonists and alpha-2 adrenergic receptor antagonists can be administered either epidurally or intrathecally. Further, as atipemazole is known to be effective by systemic administration, i.e. orally or parenterally, it is expected that systemic administration of this agent as well as other alpha-2 adrenergic receptor antagonists in combination with epidural or intrathecal administration of the alpha-2 adrenergic receptor agonist will also be effective in pain management. Further, in other applications such as hypertension, glaucoma, nasal congestion, anxiety and opioid withdrawal symptoms, alpha-2 adrenergic receptor agonists can be administered systemically or locally, and by any suitable route such as orally, intravenously, intramuscularly, intraperitoneally, topically, rectally, dermally, transdermally, subcutaneously, sublingually, buccally, intranasally, intraocularly or via inhalation. Preferably, the alpha-2 adrenergic receptor agonist and alpha-2 adrenergic receptor antagonist are administered simultaneously via the same route of administration. However, it is expected that administration of the compounds separately, via the same route or different route of administration, within a time frame during which each therapeutic compound remains active, will also be effective therapeutically as well as in alleviating tolerance to the alpha-2 adrenergic receptor agonist. Further, it is expected that administration of an alpha-2 adrenergic receptor antagonist to a subject already receiving alpha-2 adrenergic receptor agonist treatment will reverse any tolerance to the alpha-2 adrenergic receptor agonist and restore therapeutic activity, in particular analgesic potency of the alpha-2 adrenergic receptor agonist. Thus, treatment with the alpha-2 adrenergic receptor agonist and alpha-2 adrenergic receptor antagonist in the combination therapy of the present invention need not begin at the same time. Instead, administration of the alpha-2 adrenergic receptor antagonist may begin several days, weeks, months or more after treatment with the alpha-2 adrenergic receptor agonist.

Accordingly, for purposes of the present invention, the therapeutic compounds, namely the alpha-2 adrenergic receptor agonist and the alpha-2 adrenergic receptor antagonist, can be administered together in a single pharmaceutically acceptable vehicle or separately, each in their own pharmaceutically acceptable vehicle.

As used herein, the term “therapeutic compound” is meant to refer to an alpha-2 adrenergic receptor agonist and/or an alpha-2 adrenergic receptor antagonist.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compound and are physiologically acceptable to a subject. An example of a pharmaceutically acceptable vehicle is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Carrier or substituent moieties useful in the present invention may also include moieties which allow the therapeutic compound to be selectively delivered to a target organ. For example, delivery of the therapeutic compound to the brain may be enhanced by a carrier moiety using either active or passive transport (a “targeting moiety”). Illustratively, the carrier molecule may be a redox moiety, as described in, for example, U.S. Pat. Nos. 4,540,654 and 5,389,623, both to Bodor. These patents disclose drugs linked to dihydropyridine moieties which can enter the brain, where they are oxidized to a charged pyridinium species which is trapped in the brain. Thus drugs linked to these moieties accumulate in the brain. Other carrier moieties include compounds, such as amino acids or thyroxine, which can be passively or actively transported in vivo. Such a carrier moiety can be metabolically removed in vivo, or can remain intact as part of an active compound. Structural mimics of amino acids (and other actively transported moieties) including peptidomimetics, are also useful in the invention. As used herein, the term “peptidomimetic” is intended to include peptide analogues which serve as appropriate substitutes for peptides in interactions with, for example, receptors and enzymes. The peptidomimetic must possess not only affinity, but also efficacy and substrate function. That is, a peptidomimetic exhibits functions of a peptide, without restriction of structure to amino acid constituents. Peptidomimetics, methods for their preparation and use are described in Morgan et al. (1989), the contents of which are incorporated herein by reference. Many targeting moieties are known, and include, for example, asialoglycoproteins (see e.g., Wu, U.S. Pat. No. 5,166,320) and other ligands which are transported into cells via receptor-mediated endocytosis (see below for further examples of targeting moieties which may be covalently or non-covalently bound to a target molecule).

The term “subject” as used herein is intended to include living organisms in which pain to be treated can occur. Examples of subjects include mammals such as humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. As evidenced by Mordenti (1986) and similar articles, dosage forms for animals such as, for example, rats can be and are widely used directly to establish dosage levels in therapeutic applications in higher mammals, including humans. In particular, the biochemical cascade initiated by many physiological processes and conditions is generally accepted to be identical in mammalian species (see, e.g., Mattson and Scheff, 1994; Higashi et al., 1995). In light of this, pharmacological agents that are efficacious in animal models such as those described herein are believed to be predictive of clinical efficacy in humans, after appropriate adjustment of dosage.

Depending on the route of administration, the therapeutic compound may be coated in a material to protect the compound from the action of acids, enzymes and other natural conditions which may inactivate the compound. Insofar as the invention provides a combination therapy in which two therapeutic compounds are administered, each of the two compounds may be administered by the same route or by a different route. Also, the compounds may be administered either at the same time (i.e., simultaneously) or each at different times. In some treatment regimes it may be beneficial to administer one of the compounds more or less frequently than the other.

The compounds of the invention can be formulated to ensure proper distribution in vivo. For example, the blood-brain barrier (BBB) excludes many highly hydrophilic compounds. To ensure that the therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade et al., 1989). Exemplary targeting moieties include folate and biotin (see, e.g., U.S. Pat. No. 5,416,016 to Low et al.); mannosides (Umezawa et al., 1988); antibodies (Bloeman et al., 1995; Owais et al., 1995); and surfactant protein A receptor (Briscoe et al., 1995). In a preferred embodiment, the therapeutic compounds of the invention are formulated in liposomes; in a more preferred embodiment, the liposomes include a targeting moiety.

Delivery and in vivo distribution can also be affected by alteration of an anionic group of compounds of the invention. For example, anionic groups such as phosphonate or carboxylate can be esterified to provide compounds with desirable pharmocokinetic, pharmacodynamic, biodistributive, or other properties.

To administer a therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally (e.g., intramuscularly, subcutaneously intravenously, intraocularly, intraperitoneally, intraspinally, intrathecally, intracerebrally, intranasally or via inhalation). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g., vegetable oil). The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compound is mixed with at least one inert, pharmaceutically acceptable excipient or diluent or assimilable edible carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable carrier, in patch form).

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compounds according to the invention are administered at a therapeutically effective dosage sufficient to achieve the desired therapeutic effect of the alpha-2 adrenergic receptor agonist, e.g. to mitigate pain and/or to effect analgesia in a subject, to lower blood pressure, to treat glaucoma, and/or to alleviate nasal congestion, anxiety and opioid withdrawal symptoms. For example, if the desired therapeutic effect is analgesia, the “therapeutically effective dosage” mitigates pain by about 25%, preferably by about 50%, even more preferably by about 75%, and still more preferably by about 100% relative to untreated subjects. Actual dosage levels of active ingredients in the pharmaceutical compositions of this invention may be varied so as to obtain an amount of the active compound(s) that is effective to achieve and maintain the desired therapeutic response for a particular subject, composition, and mode of administration. The selected dosage level will depend upon the activity of the particular compound, the route of administration, frequency of administration, the severity of the condition being treated, the condition and prior medical history of the subject being treated, the age, sex, weight and genetic profile of the subject, and the ability of the therapeutic compound to produce the desired therapeutic effect in the subject. Dosage regimens can be adjusted to provide the optimum therapeutic response. For example, several divided doses may be administered daily or the dose may be proportionally reduced as indicated by the exigencies of the therapeutic situation.

However, it is well known within the medical art to determine the proper dose for a particular patient by the dose titration method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed. Also, dosages are routinely determined through preclinical ADME toxicology studies and subsequent clinical trials as required by the FDA or equivalent agency. The ability of an alpha-2 adrenergic receptor agonist to produce the desired therapeutic effect may be demonstrated in various well known models for the various conditions treated with these therapeutic compounds. For example, mitigation of pain can be evaluated in model systems that may be predictive of efficacy in mitigating pain in human diseases and trauma, such as animal model systems known in the art (including, e.g., the models described herein).

The following nonlimiting examples are provided to further illustrate the present invention.

### EXAMPLES

### Example 1

### Animals

Experiments were conducted using adult male Sprague-Dawley rats (Charles River, St. Constant, QC, Canada) weighing between 200-250 grams. Animals were housed individually in standard laboratory cages, maintained on a 12-hour light/dark cycle, and provided with food and water ad libitum. The surgical placement of chronic indwelling intrathecal catheters (polyethylene PE 10 tubing, 7.5 cm) into the spinal subarachnoid space was made under 4% halothane anesthesia, using the method of Yaksh and Rudy Physiol. Behav. 1976 7:1032-1036). Specifically, the anesthetized animal was placed prone in a stereotaxic frame, a small incision made at the back of the neck, and the atlanto-occipital membrane overlying the cisterna magna was exposed and punctured with a blunt needle. The catheter was inserted through the cisternal opening and slowly advanced caudally to position its tip at the lumbar enlargement. The rostral end of the catheter was exteriorized at the top of the head and the wound closed with sutures. Animals were allowed 3-4 days recovery from surgery and only those free from neurological deficits, such as the hindlimb or forelimb paralysis or gross motor dysfunction, were included in the study. All drugs were injected intrathecally as solutions dissolved in physiological saline (0.9%) through the exteriorized portion of the catheter at a volume of 10 μl, followed by a 10 μl volume of 0.9% saline to flush the catheter.

### Example 2

### Assessment of Nociception

The response to brief nociceptive stimuli was tested using two tests: the tail flick test and the paw pressure test.

The tail flick test (D'amour & Smith, J. Pharmacol. Exp. Ther. 1941 72:74-79) was used to measure the response to a thermal nociceptive stimulus. Radiant heat was applied to the distal third of the animal's tail and the response latency for tail withdrawal from the source was recorded using an analgesia meter (Owen et al., J. Pharmacol. Methods 1981 6:33-37)). The stimulus intensity was adjusted to yield baseline response latencies between 2-3 seconds. To minimize tail damage, a cutoff of 10 seconds was used as an indicator of maximum antinociception.

The paw pressure test (Loomis et al., Pharm. Biochem. 1987 26:131-139) was used to measure the response to a mechanical nociceptive stimulus. Pressure was applied to the dorsal surface of the hind paw using an inverted air-filled syringe connected to a gauge and the value at which the animal withdrew its paw was recorded. A maximum cutoff pressure of 300 mmHg was used to avoid tissue damage. Previous experience has established that there is no significant interaction between the tail flick and paw pressure tests (Loomis et al., Can. J. Physiol. Pharmacol. 1985 63:656-662).

### Example 3

### Determination of Inhibition of Clonidine Analgesia by Alpha-2 Adrenergic Receptor Antagonists

The effects of atipemazole were tested on the acute analgesic action of spinal clonidine to establish that this drug acts as an alpha-2 adrenergic receptor antagonist. A single injection of clonidine was administered intrathecally and the response measured in the tail flick and paw pressure test. In subsequent tests, clonidine was delivered in combination with 1, 5 or 10 μg atipemazole. Following drug administration, nociceptive testing was performed every 10 minutes for the first 60 minutes and every 30 minutes for the following 120-150 minute period. Results for atipemazole are depicted in FIG. 1A (tail flick) and FIG. 1B (paw pressure).

### Example 4

### Data Analysis

For the in vivo studies, tail flick and paw pressure values were converted to a maximum percentage effect (M.P.E.): M.P.E.=100×[post-drug response−baseline response]/[maximum response−baseline response]. Data represented in the figures are expressed as mean (±S.E.M.). The ED₅₀ values were determined using a non-linear regression analysis (Prism 2, GraphPad Software Inc., San Diego, Calif., USA). Statistical significance (p<0.05, 0.01. or 0.001) was determined using a one-way analysis of variance followed by a Student Newman-Keuls post hoc test for multiple comparisons between groups.

What is claimed is:

1. A composition comprising an alpha-2 adrenergic receptor agonist at a concentration effective to produce a therapeutic effect and an alpha-2 adrenergic receptor antagonist at a concentration effective to potentiate, but not antagonize, a therapeutic effect of the alpha-2 adrenergic receptor agonist.

2. The composition of claim 1 wherein the alpha-2 adrenergic receptor agonist is selected from the group consisting of L-norepinephrine, clonidine, dexmetdetomidine, apraclonidine, tizanidine, brimonidine, xylometazoline, tetrahydrozoline, oxymetazoline, guanfacine, guanabenz, xylazine, moxonidine, rilmenidine, UK 14,304, B-HT 933, B-HT 920, and octopamine.

3. The composition of claim 1 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

4. The composition of claim 1 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

5. A method for potentiating a therapeutic effect of an alpha-2 adrenergic receptor agonist in a subject, the method comprising administering an alpha-2 adrenergic receptor agonist to the subject and administering an alpha-2 adrenergic receptor antagonist to the subject, wherein the alpha-2 adrenergic receptor antagonist is at a concentration effective to potentiate, but not antagonize the therapeutic effect of the alpha-2 adrenergic receptor agonist.

6. The method of claim 5 wherein the alpha-2 adrenergic receptor agonist is selected from the group consisting of L-norepinephrine, clonidine, dexmetdetomidine, apraclonidine, tizanidine, brimonidine, xylometazoline, tetrahydrozoline, oxymetazoline, guanfacine, guanabenz, xylazine, moxonidine, rilmenidine, UK 14,304, B-HT 933, B-HT 920, and octopamine.

7. The method of claim 5 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

8. The method of claim 5 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

9. The method of claim 5 wherein the therapeutic effect of the alpha-2 adrenergic receptor agonist is potentiated without substantial side effects.

10. A method for potentiating a therapeutic effect of an endogenous alpha-2 adrenergic receptor agonist or a drug, action of which is dependent at least in part on an endogenous alpha-2-adrenergic receptor agonist in a subject, the method comprising administering to the subject an alpha-2 adrenergic receptor antagonist, wherein the alpha-2 adrenergic receptor antagonist is at a concentration effective to potentiate, but not antagonize the therapeutic effect of the endogenous alpha-2 adrenergic receptor agonist.

11. The method of claim 10 wherein the endogenous alpha-2 adrenergic receptor agonist is L-norepinephrine.

12. The method of claim 10 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

13. The method of claim 10 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

14. The method of claim 11 further comprising administering to the subject a drug, action of which is dependent at least in part on endogenous L-norepinephrine.

15. The method of claim 14 wherein the drug is a monoamine oxidase inhibitor, venlafaxine, reboxitine, a tricyclic antidepressant, tramadol, amphetamine or methylphenidate.

16. A method for inhibiting development of tolerance to a therapeutic effect of an alpha-2 adrenergic receptor agonist in a subject, the method comprising administering the alpha-2 adrenergic receptor agonist to the subject and administering an alpha-2 adrenergic receptor antagonist to the subject, wherein the alpha-2 adrenergic receptor antagonist is at a concentration effective to potentiate, but not antagonize the therapeutic effect of the alpha-2 adrenergic receptor agonist.

17. The method of claim 16 wherein the alpha-2 adrenergic receptor agonist is selected from the group consisting of L-norepinephrine, clonidine, dexmetdetomidine, apraclonidine, tizanidine, brimonidine, xylometazoline, tetrahydrozoline, oxymetazoline, guanfacine, guanabenz, xylazine, moxonidine, rilmenidine, UK 14,304, B-HT 933, B-HT 920, and octopamine.

18. The method of claim 16 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

19. The method of claim 16 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

20. The method of claim 16 wherein the tolerance is acute tolerance.

21. The method of claim 16 wherein the tolerance is chronic tolerance.

22. A method for reversing tolerance to a therapeutic effect of an alpha-2 adrenergic receptor agonist or restoring a therapeutic effect of an alpha-2 adrenergic receptor agonist in a subject, the method comprising administering to the subject an alpha-2 adrenergic receptor antagonist, wherein the alpha-2 adrenergic receptor antagonist is at a concentration effective to potentiate, but not antagonize the therapeutic effect of the alpha-2 adrenergic receptor agonist.

23. The method of claim 22 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

24. The method of claim 22 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

25. A method for treating a subject suffering from a condition treatable with an alpha-2 adrenergic receptor agonist, the method comprising administering an alpha-2 adrenergic receptor agonist to the subject at a concentration effective to produce a therapeutic effect and administering an alpha-2 adrenergic receptor antagonist to the subject, wherein the alpha-2 adrenergic receptor antagonist is at a concentration effective to potentiate, but not antagonize, the therapeutic effect of the alpha-2 adrenergic receptor agonist.

26. The method of claim 25 wherein the alpha-2 adrenergic receptor agonist is selected from the group consisting of L-norepinephrine, clonidine, dexmetdetomidine, apraclonidine, tizanidine, brimonidine, xylometazoline, tetrahydrozoline, oxymetazoline, guanfacine, guanabenz, xylazine, moxonidine, rilmenidine, UK 14,304, B-HT 933, B-HT 920, and octopamine.

27. The method of claim 25 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

28. The method of claim 25 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

29. The method of claim 25 wherein the subject is suffering from pain, hypertension, glaucoma, nasal congestion, anxiety or opioid withdrawal symptoms or is in need of an adjunct to peripheral nerve block.

30. The method of claim 25 wherein the subject is treated for a condition treatable with an alpha-2 adrenergic receptor agonist without substantial side effects.

31. A method for treating a subject suffering from a condition treatable with an alpha-2 adrenergic receptor agonist comprising administering to a subject receiving alpha-2 adrenergic receptor agonist therapy an alpha-2 adrenergic receptor antagonist at a concentration effective to potentiate, but not antagonize the therapeutic effect of the alpha-2 adrenergic receptor agonist.

32. The method of claim 31 wherein the alpha-2 adrenergic receptor antagonist is selected from the group consisting of atipemazole (or atipamezol), fipamazole (fluorinated derivative of atipemazole), mirtazepine (or mirtazapine), eferoxan, idozoxan (or idazoxan), Rx821002 (2-methoxy-idozoxan), rauwolscine, MK 912, SKF 86466, SKF 1563 and yohimbine.

33. The method of claim 31 wherein the alpha-2 adrenergic receptor antagonist is selected from the group consisting of venlafaxine, doxazosin, phentolamine, dihydroergotamine, ergotamine, phenothiazines, phenoxybenzamine, piperoxane, prazosin, tamsulosin, terazosin, and tolazoline.

34. The method of claim 31 wherein the subject is suffering from pain, hypertension, glaucoma, nasal congestion, anxiety or opioid withdrawal symptoms or is in need of an adjunct to peripheral nerve block.

35. The method of claim 31 wherein the subject is treated for a condition treatable with an alpha-2 adrenergic receptor agonist without substantial side effects.
